(19) United States
(12) Patent Application Publication
(10) Pub. No.: US 2024/0260858 A1
(43) Pub. Date: Aug. 8, 2024

(54) SIMULTANEOUS AND CONTINUOUS MEASUREMENT OF OTOACOUSTIC EMISSIONS ACROSS LEVEL AND FREQUENCY

(71) Applicants: Northwestern University, Evanston, IL (US); University of Iowa Research Foundation, Iowa City, IA (US)

(72) Inventors: Sumitrajit Dhar, Wilmette, IL (US); Shawn Goodman, Iowa City, IA (US); Courtney Shea Coburn Glavin, Chicago, IL (US)

(21) Appl. No.: 18/434,313

(22) Filed: Feb. 6, 2024

Related U.S. Application Data
(60) Provisional application No. 63/483,328, filed on Feb. 6, 2023.

(51) Int. Cl. A61B 5/12; G16H 50/30

(52) U.S. Cl. CPC A61B 5/126; G16H 50/30

(57) ABSTRACT

A method of conducting an otoacoustic emission test includes emitting, by a signal generator, a stimulus tone that is to be received by a user, where the stimulus tone comprises a swept level stimuli. The method also includes recording, in a memory of a computing device, otoacoustic emissions generated by an inner ear of the user, where the otoacoustic emissions are in response to the stimulus tone. The method further includes determining, by a processor of the computing device, one or more health characteristics of the inner ear of the user based at least in part on the recorded otoacoustic emissions.

### CROSS-REFERENCE TO RELATED APPLICATION

The present application claims the priority benefit of U.S. Provisional Patent App. No. 63/483,328 filed on Feb. 6, 2023, the entire disclosure of which is incorporated by reference herein.

### REFERENCE TO GOVERNMENT RIGHTS

This invention was made with government support under grant number DC019557 awarded by the National Institutes of Health. The government has certain rights in the invention.

### BACKGROUND

An otoacoustic emission (OAE) test refers to a test that is used to determine how well an individual's inner ear (as opposed to the middle/outer ear) works. A typical OAE test measures otoacoustic emissions, which are sounds that are given off by the inner ear when the inner ear responds to a sound. More specifically, the inner ear includes hair cells that respond to sound by vibrating. These vibrations produce a sound that echoes back into the individual's middle ear, and this sound represents the OAEs.

### SUMMARY

An illustrative method of conducting an otoacoustic emission test includes emitting, by a signal generator, a stimulus tone that is to be received by a user, where the stimulus tone comprises a swept level stimuli. The method also includes recording, in a memory of a computing device, otoacoustic emissions generated by an inner ear of the user, where the otoacoustic emissions are in response to the stimulus tone. The method further includes determining, by a processor of the computing device, one or more health characteristics of the inner ear of the user based at least in part on the recorded otoacoustic emissions.

In an illustrative embodiment, the swept level stimuli simultaneously includes a plurality of distinct frequencies. In another embodiment, the stimulus tone comprises a pair of stimulus tones that have a fixed frequency ratio. In one embodiment, the fixed frequency ratio is in a range between 1.1 and 1.3. In another embodiment, the method includes calibrating the stimulus tone using a forward pressure level (FPL) technique.

In one embodiment, determining the one or more health characteristics includes determining an amount of cochlear aging in the user. In another embodiment, determining the one or more health characteristics includes determining a growth function for the inner car of the user. In an illustrative embodiment, the growth function comprises a pattern of distortion product otoacoustic emission growth with increasing stimulus level at a given frequency. In one embodiment, the determining includes analyzing the recorded otoacoustic emissions using sliding time analysis windows with a weighted least-squares fitting (WLSF) procedure. In another embodiment, the sliding time analysis windows are between 100-300 milliseconds in length, and there is an overlap between windows.

An illustrative system for conducting an otoacoustic emission test includes a signal generator that emits a stimulus tone that is to be received by a user, where the stimulus tone comprises a swept level stimuli. The system also includes a computing device operatively coupled to the signal generator, wherein the computing device includes a memory that stores otoacoustic emissions generated by an inner ear of the user. The otoacoustic emissions are in response to the stimulus tone. The computing device also includes a processor operatively coupled to the memory, where the processor determines one or more health characteristics of the inner car of the user based at least in part on the otoacoustic emissions.

In an illustrative embodiment, the swept level stimuli simultaneously includes a plurality of distinct frequencies. In another embodiment, the stimulus tone comprises a pair of stimulus tones that have a fixed frequency ratio. In another embodiment, the fixed frequency ratio is in a range between 1.1 and 1.3. In one embodiment, the processor is further configured to calibrate the stimulus tone using a forward pressure level (FPL) technique.

The system can also include a measurement probe configured to record the otoacoustic emissions generated by the inner car of the user. In another embodiment, the one or more health characteristics include an amount of cochlear aging in the user. In another embodiment, the one or more health characteristics include a growth function for the inner car of the user, where the growth function comprises a pattern of distortion product otoacoustic emission growth with increasing stimulus level at a given frequency. In another embodiment, the processor analyzes the otoacoustic emissions using sliding time analysis windows with a weighted least-squares fitting (WLSF) procedure to determine the one or more health characteristics.

Other principal features and advantages of the invention will become apparent to those skilled in the art upon review of the following drawings, the detailed description, and the appended claims.

### BRIEF DESCRIPTION OF THE DRAWINGS

Illustrative embodiments of the invention will hereafter be described with reference to the accompanying drawings, wherein like numerals denote like elements.

FIG. 1 displays DPOAE growth functions from each participant measured using both paradigms (discrete and swept level) at each test frequency in accordance with an illustrative embodiment.

FIG. 2 depicts average DPOAE growth functions measured with discrete and swept level stimuli yield equivalent results at four f₂ frequencies in accordance with an illustrative embodiment.

FIG. 3 depicts average magnitude differences between DPOAE growth functions measured discretely vs. those measured with swept level stimuli at each f₂ frequency (difference=discrete−swept) in accordance with an illustrative embodiment.

FIG. 4 depicts an analysis of repeated swept level DPOAEs for one representative participant in accordance with an illustrative embodiment.

FIG. 5 depicts a system that includes a computing device for performing DPOAE analysis in accordance with an illustrative embodiment.

### DETAILED DESCRIPTION

Otoacoustic emissions (OAEs) are low-level sounds generated by the inner ear that provide a non-invasive assessment of cochlear health. Advanced applications involve recording OAEs across a wide range of frequencies and stimulus levels. Described herein are methods and systems for efficiently measuring distortion product OAEs (DPOAEs) across an expansive stimulus space. Specifically, DPOAEs are recorded by sweeping an evoking stimuli in level across multiple frequencies simultaneously. This method generates DPOAE growth functions at multiple f₂ frequencies in several minutes. Results indicate the swept level method yields DPOAEs equivalent to those measured in a traditional (discrete stimulus) paradigm, but with several advantages.

Otoacoustic emissions (OAEs) are commonly used in clinical applications as a screening tool to detect the presence or absence of hearing loss. However, advanced applications aim to provide a more comprehensive assessment of cochlear function. This may necessitate assessing OAEs over a wider range of stimulus conditions. For example, DPOAE growth functions (i.e., the pattern of DPOAE growth with increasing stimulus level at a given frequency) are sensitive to subclinical cochlear aging. Additionally, cochlear insults, including aging, tend to first affect the highest frequencies of hearing. This indicates the need to measure OAEs across a broad range of levels and frequencies to obtain a full picture of cochlear health and function.

In the most common conventional paradigm, DPOAE growth functions are measured by playing a pair of stimulus tones at adjacent frequencies at one level or at a level combination. Responses to the discrete stimuli are recorded until the signal of interest (OAE) can be extracted from the noise with confidence. This process is then repeated, as desired, using various combinations of stimuli to obtain information across level(s) and frequency range(s) of interest.

Discrete recording paradigms likely suffice when using OAEs as a screening tool (i.e., recording at only one level combination and a limited number of frequencies). However, inefficiencies may arise when attempting to record OAE growth functions (which necessitate measurement at many level combinations). First, depending on the number and range of discrete stimulus level(s) tested, the data may be too sparse to accurately capture the true nature of an individual's OAE growth. Various features of OAE growth functions, including threshold, points of maximum strength or gain, and inflection points and/or slopes have been measured. These extracted features have subsequently been related to cochlear pathology, psychophysical phenomena, and other demographic characteristics (e.g., age). Independent growth functions of DPOAE components (distortion and reflection) have also been measured using brief pulsed stimuli. Capturing the true shape of the growth function is of particular concern given that the discrete measurement levels are typically selected a priori, and that there is significant variability in OAE growth between individuals—even among those who are similar demographically.

Second, and relatedly, discrete measurement paradigms may compromise OAE growth function goodness of fit across individuals. This is particularly the case when using models with fewer parameters. In a previous analysis, the inventors used a three-segment piecewise linear fitting to characterize DPOAE growth functions. While most growth functions could be fit with this technique (566/568), up to 10% of the generated fittings were poor (i.e., did not closely match the measured data). This was true even with densely sampled growth functions measured using a relatively small stimulus level step size (e.g., 2 decibels (dB)). Analysis has shown that larger step sizes between discrete stimuli further limit the ability to accurately fit OAE growth functions with more easily interpretable models.

Described below is a method to continuously vary the level of tonal stimuli used to evoke DPOAEs and to present these swept level stimuli at multiple frequency combinations simultaneously. The method was verified by demonstrating that DPOAEs recorded using swept level stimuli are equivalent to those measured using a discrete stimulus (i.e., traditional) paradigm. The proposed swept level paradigm is an efficient method for measuring DPOAE growth, as it can yield near-continuous growth functions at multiple f₂ frequencies in several minutes.

A study was conducted to verify the above-discussed principles. Participants in the study included six adults (F=6; age range: 16-35 years) with self-reported normal hearing and audiometric thresholds≤20 dB HL from 0.25-8 KHz bilaterally. Two participants self-identified their race as Asian, three self-identified as White, and one chose not to disclose. No participants self-reported their ethnicity as Hispanic or Latino. All participants consented to participate in this research.

In the study, all DPOAEs were recorded from a randomly selected test car of each participant. A screening protocol was run to assess the presence and robustness of DPOAEs in the participant's test ear from f₂=1-10 kHz at one stimulus level combination (65/55 dB forward pressure level (FPL). The FPL is defined as a measure of the pressure level of the sum of all incident waves of the stimuli arriving at the eardrum, before growth functions were recorded. The screening protocol used a pair of stimulus tones, fixed in level and frequency ratio, and continuously swept in frequency. Participants were included in the experiment only if their DPOAEs were sufficiently above the noise floor (≥3 dB) across all frequencies in this screening. If DPOAE growth functions were measured from a participant across multiple test sessions, the screener was repeated at each session to ensure adequate test-retest reliability.

With respect to the instrumentation and calibration, audiometric thresholds from 0.25-8 kHz (at octave and inter-octave frequencies) were measured using Shoebox Audiometry in a double-walled sound-treated booth. Shoebox Audiometry is automated and self-administered by the participant using a computing device (e.g., iPad) and E-A-RTONE 3A transducers coupled to the ear with foam tips. DPOAEs were measured using an ER10X probe system (Interacoustics, Denmark; Etymotic, Elk Grove Village, IL). DPOAE signal generation and data collection were controlled using custom software (ARLas, Shawn S. Goodman) written in Matlab on a PC running Windows 10. An RME Fireface UCX II 24-bit audio interface was used for A/D and D/A conversion. DPOAE stimuli were calibrated using a forward pressure level (FPL) technique. Recorded OAEs are reported in emitted pressure level (EPL). Both procedures are designed to minimize the effects of ear canal standing waves on OAEs. In alternative embodiments, different instrumentation and/calibration techniques may be used.

DPOAE growth functions were measured using both discrete and swept level stimuli in each participant's test ear at f₂=1, 4, 8 and/or 10 kHz. For a given recording condition, f₁ was set according to either f₂/f₁=1.22 or f₂/f₁=1.2 or 1.16 at f₂=1, 4, 8, and 10 kHz, respectively. These f₂/f₁ ratios were selected to represent common clinical protocols (1.22 ratio) and to optimize DPOAE levels based on known mechanical properties of the cochlea. DPOAEs obtained using discrete and swept level stimuli were only compared across equivalent f₂/f₁ ratio conditions.

To obtain a growth function, L₁ was fixed at 70 dB FPL. L₂ was varied from 10-70 dB FPL in 10 dB steps (discrete stimuli) or swept from 0-70 dB FPL at a rate of 10 dB/second (swept level stimuli). DPOAE growth functions measured using a fixed L₁ and varying L₂ most closely match analogous growth functions in basilar membrane displacement in a guinea pig. Additionally, this stimulus paradigm has been demonstrated to yield DPOAE growth functions that are sensitive to subclinical signs of cochlear aging.

Discrete stimuli were 1 second in duration. Each buffer included three stimulus presentations and was repeated 32 times at each test condition. Swept level stimuli were 7 seconds in duration (before ramping and zero-padding) and were repeated 40 times per test condition. For discrete stimuli, test length for each level and frequency combination (e.g., 70/70 at f₂=1 kHz) was approximately 2-3 minutes. Thus, measurement of a coarse (10 dB step size) DPOAE growth function at a single f₂ frequency took approximately 15 minutes. In contrast, multiple DPOAE growth functions measured using swept level stimuli could be obtained in less than 4 minutes (when measuring at multiple f₂ frequencies simultaneously). That is, the growth functions measured using discrete level stimuli were obtained while presenting a single pair of tones during a given measurement window. In contrast, growth functions for swept level tones were obtained using multiple pairs of tones presented simultaneously at different frequencies. For example, the growth function for f₂=1 kHz was recorded in isolation as well as with growth functions at one or more test frequencies (f₂=4, 8, & 10 kHz). It is acknowledged that parameter settings (e.g., stimulus length, number of repetitions) can be varied to impact overall test time. These parameters should be selected according to the application. These concepts are further discussed in more detail below.

Data recorded using discrete stimuli were analyzed using a fast Fourier transform (FFT). Data recorded using the swept level method were analyzed using sliding time analysis windows (200 ms in length, ˜17 ms of overlap between windows) with a weighted least-squares fitting (WLSF) procedure. Alternatively, the windows can be anywhere between 100-300 ms in length, and the amount of overlap can be different (e.g., 10-30 ms). The ordinary LSF procedure for estimating the 2f₁−f₂ total DPOAE is known in the art. In short, the LSF procedure scales a model DPOAE response to minimize the sum of squares error between the model and measured pressure. To fit data collected with swept level stimuli, the inventors used sinusoidal basis functions of unit amplitude multiplied by a Blackman window. For swept stimuli, the WLSF procedure is preferable because it allows noisy samples to be removed from the analysis without discarding the entire sweep in which they occurred. Weighting values of zero were assigned to noisy time samples, identified by considering the variance across repeated sweeps. For each sweep, the WLSF model was used to obtain coefficients for the cosine- and sine-phase portions of the model DPOAE. The coefficients were combined into complex form (similar to complex Fourier coefficients). The DPOAE signal was then calculated as the magnitude of the mean of the complex coefficients across repeated sweeps (i.e., across stimulus repetitions). The noise floor (for both discrete and swept level data) was calculated as the standard error of the mean across repeated sweeps.

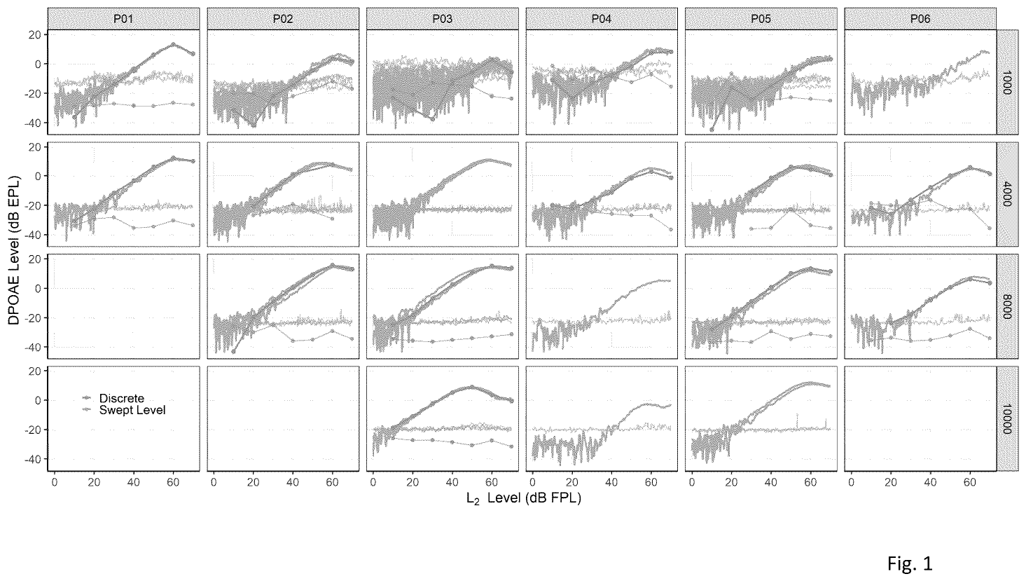

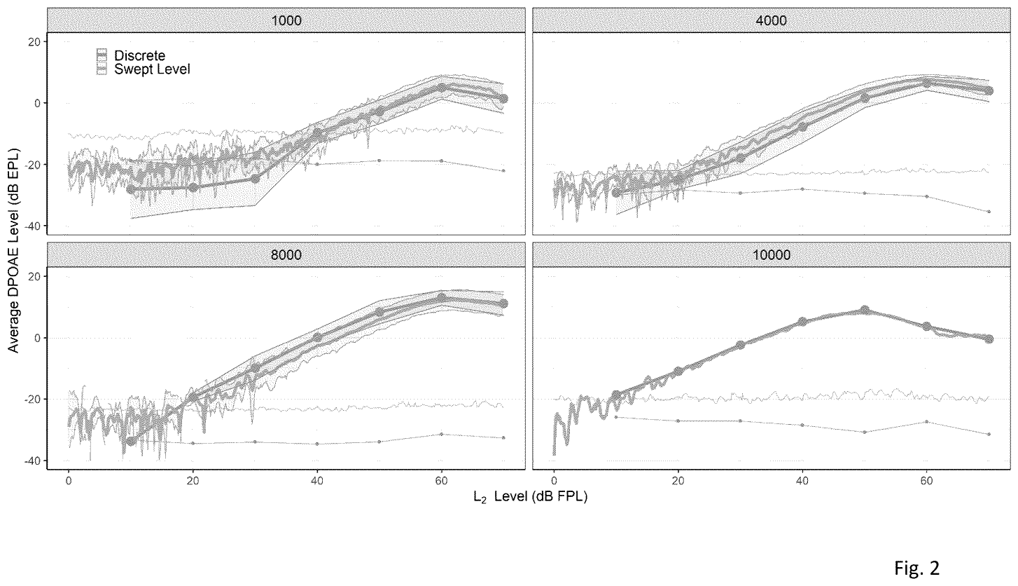

DPOAE growth functions measured using swept level stimuli closely resembled those measured using discrete tones. This is highlighted across individual participants in FIG. 1 and on average in FIG. 2 Specifically, FIG. 1 displays DPOAE growth functions from each participant measured using both paradigms (discrete and swept level) at each test frequency in accordance with an illustrative embodiment. Individual DPOAE growth functions were measured with discrete (lines connected by dots) and swept level (shaded area) stimuli yield equivalent results at four f₂ frequencies across participants. Associated noise floors for each stimulus paradigm are also shown in FIG. 1. Blank panels indicate that a participant did not have measured data at that particular f₂ frequency. Panels missing discrete data indicate that discrete data could not be compared to swept level data for that participant/frequency combination because discrete data were not measured with an optimal f₂/f₁ ratio.

Some participants had multiple growth functions measured within a given stimulus/frequency condition (all of which are shown), while others did not have a growth function for a given condition (indicated by blank panels). Only data with matched f₂/f₁ ratios between stimulus conditions are shown in FIG. 1. For each participant, both stimulus conditions yielded similar DPOAE estimates. While there are noticeable differences in noise floors between stimulus conditions, these are primarily due to differences in averaging time. Specifically, the noise floors estimated from the discrete stimulus condition tend to be lower because of longer averaging times. This is discussed in more detail below.

FIG. 2 depicts average DPOAE growth functions measured with discrete and swept level stimuli yield equivalent results at four f₂ frequencies in accordance with an illustrative embodiment. Associated noise floors for each stimulus paradigm are also shown. The shaded regions around each DPOAE growth curve represent twice the standard error of the mean. One growth function from each stimulus condition for each participant was chosen for averaging. It is noted that f₂=10 KHz has data from only one participant.

Still referring to FIG. 2, average DPOAE growth functions obtained with both paradigms at each f₂ frequency with 95% confidence intervals are shown. To compare average differences between conditions, one DPOAE growth function obtained using each measurement paradigm (discrete and swept) was selected from each participant per f₂ frequency. In the cases where participants had multiple discrete or swept growth functions at a given f₂ frequency, one from each condition was selected randomly. In cases where participants did not have a growth function for either or both conditions at a given f₂ frequency, they were excluded from the average calculation at that f₂ frequency. Only growth functions with matched f₂/f₁ ratios within a given participant between stimulus conditions were used to compare average differences. Prior to averaging, growth function data from each participant were cleaned by removing DPOAE data points that were less than 3 dB above the noise floor. It is noted that data are available from only one participant for f₂=10 kHz, primarily because all other participants had swept data collected with f₂/f₁=1.16 while discrete data were collected with f₂/f₁=1.22. This resulted in differences between growth functions that can be attributed to mechanical properties of the cochlea rather than measurement paradigm (not shown). Therefore, those growth functions were excluded from analysis here. In alternative embodiments, a different frequency ratio may be used. For example, the frequency ratio may be in the range of 1.10-1.30, etc.

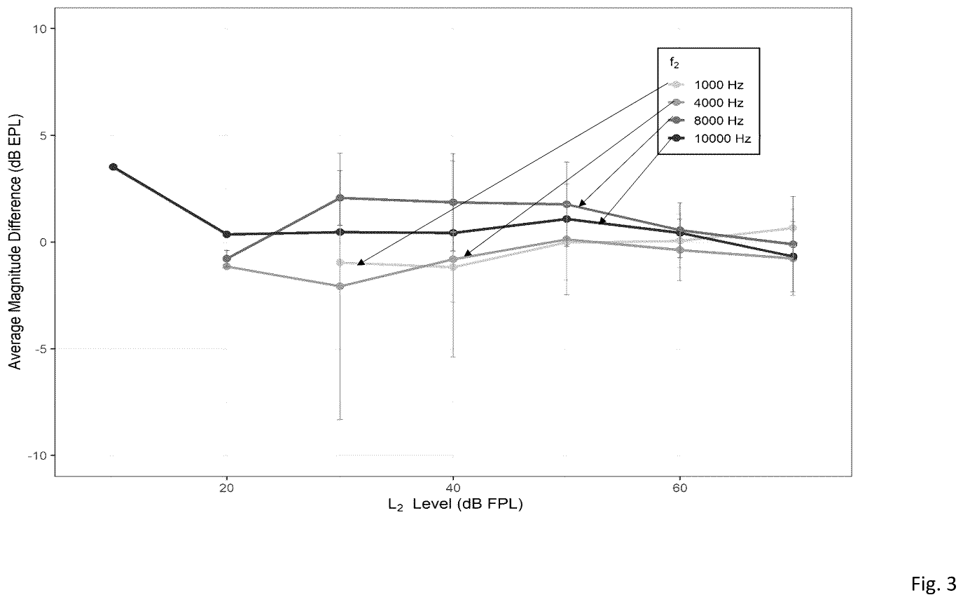

FIG. 3 depicts average magnitude differences between DPOAE growth functions measured discretely vs. those measured with swept level stimuli at each f₂ frequency (difference=discrete−swept) in accordance with an illustrative embodiment. DPOAE data for each participant at a given level were included in the average calculation if they met an signal-to-noise (SNR) criterion of >6 dB in both measurement paradigms (discrete and swept). A positive value indicates that the discretely measured DPOAE was higher in level. Error bars indicate +/−1 standard deviation of the mean. Points without error bars indicate that data from only one participant are shown. Average differences between the two stimulus conditions are <5 dB at all frequencies.

As shown in FIG. 3, average differences between DPOAEs obtained with the two measurement paradigms (discrete vs. swept) were within +/−5 dB across all test frequencies. Therefore, differences between stimulus conditions are consistent with typical test-retest reliability expected for OAE measurement at all f₂ frequencies tested. To be included in the average difference calculation, DPOAE data from a given participant at a given level were required to have an SNR>6 dB in both measurement paradigms. Maximal differences between stimulus conditions—particularly those greater than 5 dB—tended to occur at lower L₂ levels (L₂<˜30 dB FPL). This is where the signal-to-noise ratio (SNR) of measured DPOAEs tended to be the smallest for both stimulus types before cleaning the data for SNR. Therefore, it is expected that differences between conditions are greatest here, as test-retest reliability is likely to be worse (higher) when SNR is poorer. Notably, variability between conditions was not higher near f₂ frequencies typically impacted by standing waves (e.g., f₂=4 kHz and 10 kHz), reaffirming the advantages of FPL and EPL calibration techniques.

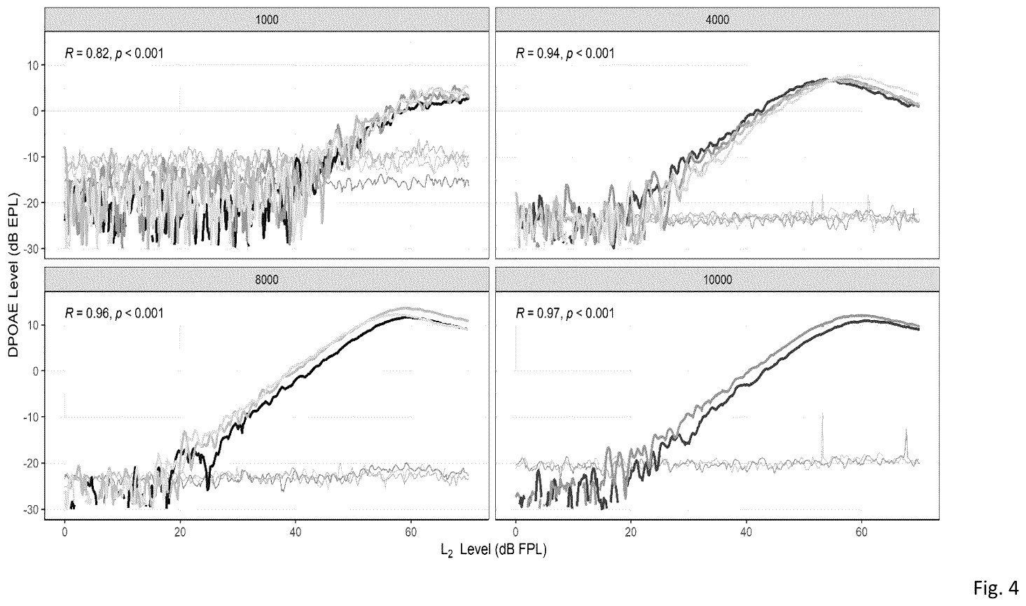

To explore the repeatability of DPOAEs obtained using swept level stimuli, swept growth functions were repeated across sessions in a subset of participants (n=2). This subset of participants was selected for follow-up measures based on their availability to return for additional testing. In these participants, swept level DPOAEs were measured two or more times at a given f₂ frequency. FIG. 4 depicts an analysis of repeated swept level DPOAEs for one representative participant in accordance with an illustrative embodiment. Each line/curve in FIG. 4 indicates the run number and therefore shows which f₂ frequencies were measured simultaneously (e.g., the lines in the 1, 4, and 8 kHz panels indicate that data from these three f₂ frequencies were measured together). These growth functions were obtained across probe insertions and test sessions. The data displayed from this participant includes measures across various probe insertions and test sessions. It also includes DPOAE growth functions measured with various simultaneously presented f₂ stimulus combinations. Pearson correlation coefficients (shown in each panel) were calculated at each frequency for this participant and indicated strong associations between sweeps—and therefore high test-retest reliability—at all frequencies: 1 kHz (r(407)=0.82, p<0.001), 4 kHz (r(407)=0.94, p<0.001), 8 kHz (r(407)=0.96, p<0.001), and 10 kHz (r(407)=0.97, p<0.001).

As previously stated, swept level data was also measured with either two, three, or four f₂ and f₁ frequency combinations presented simultaneously. Thus, for example, the frequency condition f₂=1 kHz may have been measured simultaneously with 1-2 other frequency conditions (e.g., f₂=1 & 8 kHz, or f₂=1, 4, & 8 kHz, or f₂=1, 4, 8, & 10 kHz). This was done as a proof of concept to verify that multiple frequency combinations tested simultaneously would yield equivalent results, and therefore could reduce recording time even further than a single swept level stimulus alone. All combinations of stimuli yielded equivalent results except when f₂=8 kHz and 10 kHz were presented simultaneously. In this condition, the DPOAE growth function at f₂=8 kHz was reduced, indicative of two-tone suppression (not shown).

Included below is a discussion of the proposed system and an analysis of the test results. Since the discovery of OAEs, decades of research has highlighted their clinical potential. In particular, recent work has shown the potential of using OAEs to detect subclinical aging, to differentiate between cochlear pathologies, and to predict behavioral thresholds. However, these demonstrations in the laboratory have not translated to clinical practice, where OAEs continue to be used primarily as a screening tool to detect the presence or absence of hearing loss. In part, this is because using OAEs for more advanced applications requires increasing the stimulus space over which they are measured. The inventors have therefore focused on more efficient measurement of OAEs, which has led to significant time-savings by using stimuli swept in frequency. The use of stimuli swept in level presented simultaneously at multiple f₂ frequencies for the efficient recording of DPOAE growth functions is validated herein.

The test results show that DPOAEs measured using swept level stimuli are equivalent to those measured using traditional discrete stimuli, and that DPOAEs obtained using this method are highly repeatable. Thus, using swept level stimuli to measure DPOAE growth functions is both a valid and reliable approach. This approach may be particularly useful when attempting to efficiently determine the overall shape of an individual's growth function(s), which can vary widely across ears and may not be fully captured when sampling a growth function using discrete levels that are selected without prior knowledge of an individual's OAE growth. Additionally, a nearly continuous growth function may have the added advantage of improving the goodness of fit of post hoc models used to characterize it, especially when fitting a model with fewer parameters.

Slight discrepancies between the stimulus conditions were noted herein, including a systematic trend of discretely measured noise floors being lower than noise floors estimated from swept level stimuli. This is due to differences in the measurement techniques between stimulus conditions. Specifically, discrete stimuli were 1 second in duration and repeated 96 times at a given L₁/L₂ combination. This is equivalent to 96 seconds of stimulus presentation (and therefore averaging) at a given discrete point along the function. In contrast, the swept level stimuli varied from 0 to 70 dB FPL at a rate of 10 dB/s, and was repeated 40 times. Thus, while the duration of one stimulus was longer (7 seconds), it covered a much broader range of L₂ levels. It was therefore equivalent to ˜0.68 seconds (0.017 seconds*40 repetitions) of stimulus presentation/averaging at a given discrete point along the growth function. Increased averaging is expected to decrease the estimated noise floor. Since the difference between estimated DPOAEs between the two conditions is within the range of expected test-retest reliability of OAEs and noise floor differences could be further minimized by changing the recording parameters of swept stimuli (i.e., by changing the number of repetitions, reducing the sweep rate, and/or reducing the range of levels), this is not an underlying problem with swept level stimuli. These recording parameters can and should be varied depending on the application. For example, if estimating DPOAE growth function thresholds is a priority, averaging should be increased—at least at stimulus levels near the expected threshold.

A potential limitation of using swept level stimuli is the inability to separate DPOAE components using common approaches such as time-windowing. Notably, most of the growth functions measured with swept level stimuli are relatively smooth across levels. This lack of fine structure implies that the distortion component may be mostly dominant, at least in the cars tested in the above-discussed study. This could be due, in part, to holding L₁ at a constant 70 dB FPL, thereby limiting the phase variation between DPOAEs generated by adjacent generators. This limitation can be overcome by using pulsed stimulus tones that allow temporal separation of DPOAE components and therefore independent growth functions for DPOAE components.

In addition to the gains of using swept level stimuli, the feasibility of simultaneous stimulation with multiple frequency pairs was also demonstrated. This process results in several growth functions in a brief period. Practically, the analysis suggests that f₂ frequencies should be spaced appropriately far apart so as to not lead to tone-on-tone suppression when recording multiple growth functions simultaneously. As noted, the DPOAE growth function(s) measured at f₂=8 kHz were lower when measured simultaneously with the f₂=10 KHz stimulus combination. This is likely because when f₂=10 kHz, f₁=8.62 kHz with the stimulus parameters (f₂/f₁=1.16). Thus, f₁ was close enough and high enough in level (L₁=70 dB FPL) to suppress f₂=8 kHz. While additional testing can be performed to determine optimal stimulus spacing, it is believed that setting f₂ frequencies a minimum of one octave apart should be a sufficient starting point. If measuring across the frequency range of human hearing, this would allow for up to six L₂ sweeps (e.g., f₂=0.5, 1, 2, 4, 8, and 16 kHz) simultaneously. In an alternative embodiment, stimulus spacing can be reduced further (e.g., half of an octave apart).

Additionally, while this description focuses on recording DPOAE growth functions using swept level stimuli, this measurement paradigm could be extended. For example, DPOAE growth functions could also be obtained by using multiple swept level stimuli presented simultaneously to co-vary in level (i.e., the “Scissors” paradigm). Swept level stimuli could also be used to obtain other types of OAE growth functions, including stimulus frequency OAEs (SFOAEs).

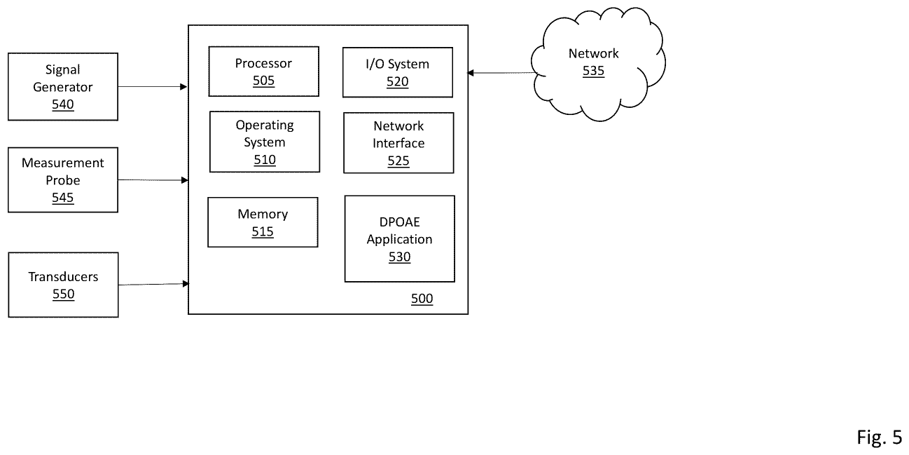

In an illustrative embodiment, any of the operations or calculations described herein can be performed by a computing system that includes a processor, a memory, a user interface, a transceiver (receiver and/or transmitter), etc. The operations can be stored as computer-readable instructions in the memory. Upon execution by the processor of the computer-readable instructions, the computing system performs the operations, calculations, etc. described herein. As an example, FIG. 5 depicts a system that includes a computing device 500 for performing DPOAE analysis in accordance with an illustrative embodiment. In an illustrative embodiment, the computing device 500 can be incorporated into a system that includes all of the components of FIG. 5. Alternatively, the computing device 500 can be separate from the remaining components of the system, but in communication therewith through a network 535 and/or through a direct wired connection.

The computing device 500 includes a processor 505, an operating system 510, a memory 515, an input/output (I/O) system 520, a network interface 525, and a DPOAE application 530. In alternative embodiments, the computing device 500 may include fewer, additional, and/or different components. The components of the computing device 500 communicate with one another via one or more buses or any other interconnect system. The system also includes a signal generator 540, a measurement probe 545, and transducers 550.

The processor 505 can be in electrical communication with and used to control any of the system components described herein. For example, the processor 505 can be used to execute the DPOAE application 530, control the signal generator 540, control the measurement probe 545, control the transducers 550, perform calculations, send/receive results, etc. The processor 505 can be any type of computer processor known in the art, and can include a plurality of processors and/or a plurality of processing cores. The processor 505 can include a controller, a microcontroller, an audio processor, a hardware accelerator, a digital signal processor, etc. Additionally, the processor 505 may be implemented as a complex instruction set computer processor, a reduced instruction set computer processor, an x86 instruction set computer processor, etc. The processor 505 is used to run the operating system 510, which can be any type of operating system.

The operating system 510 is stored in the memory 515, which is also used to store programs, received measurements/data, network and communications data, peripheral component data, the DPOAE application 530, and other operating instructions. The memory 515 can be one or more memory systems that include various types of computer memory such as flash memory, random access memory (RAM), dynamic (RAM), static (RAM), a universal serial bus (USB) drive, an optical disk drive, a tape drive, an internal storage device, a non-volatile storage device, a hard disk drive (HDD), a volatile storage device, etc. In some embodiments, at least a portion of the memory 515 can be in the cloud to provide cloud storage for the system. Similarly, in one embodiment, any of the computing components described herein (e.g., the processor 505, etc.) can be implemented in the cloud such that the system can be run and controlled through cloud computing.

The I/O system 520 is the framework which enables users and peripheral devices to interact with the computing device 500. The I/O system 520 can include one or more buttons or other controls, etc. that allow the user to interact with and control the computing device 500. The I/O system 520 also includes circuitry and a bus structure to interface with peripheral computing devices such as the imaging sensor, power sources, universal service bus (USB) devices, data acquisition cards, peripheral component interconnect express (PCIe) devices, serial advanced technology attachment (SATA) devices, high definition multimedia interface (HDMI) devices, proprietary connection devices, etc.

The network interface 525 includes transceiver circuitry (e.g., a transmitter and a receiver) that allows the computing device 500 to transmit and receive data to/from other devices such as remote computing systems, servers, websites, etc. The network interface 525 enables communication through the network 535, which can be one or more communication networks. The network 535 can include a cable network, a fiber network, a cellular network, a wi-fi network, a landline telephone network, a microwave network, a satellite network, etc. The network interface 525 also includes circuitry to allow device-to-device communication such as Bluetooth® communication.

The DPOAE application 530 can include software and algorithms in the form of computer-readable instructions which, upon execution by the processor 505, performs any of the various operations described herein such as controlling emission of stimulus tones, controlling the frequency, timing, and duration of the stimulus tones, controlling the measurement probe to detect otoacoustic emissions from the inner car, controlling the transducers, processing detected otoacoustic emissions, determining health characteristics/risks to the user based on the processing, etc. The DPOAE application 530 can utilize the processor 505 and/or the memory 455 as discussed above. In an alternative implementation, the DPOAE application 530 can be remote or independent from the computing device 500, but in communication therewith.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more.”

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed is:

1. A method of conducting an otoacoustic emission test, the method comprising:
emitting, by a signal generator, a stimulus tone that is to be received by a user, wherein the stimulus tone comprises a swept level stimuli;
recording, in a memory of a computing device, otoacoustic emissions generated by an inner ear of the user, wherein the otoacoustic emissions are in response to the stimulus tone; and
determining, by a processor of the computing device, one or more health characteristics of the inner ear of the user based at least in part on the recorded otoacoustic emissions.

2. The method of claim 1, wherein the swept level stimuli simultaneously includes a plurality of distinct frequencies.

3. The method of claim 1, wherein the stimulus tone comprises a pair of stimulus tones that have a fixed frequency ratio.

4. The method of claim 3, wherein the fixed frequency ratio is in a range between 1.1 and 1.3.

5. The method of claim 1, further comprising calibrating the stimulus tone using a forward pressure level (FPL) technique.

6. The method of claim 1, wherein determining the one or more health characteristics includes determining an amount of cochlear aging in the user.

7. The method of claim 1, wherein determining the one or more health characteristics includes determining a growth function for the inner ear of the user.

8. The method of claim 7, wherein the growth function comprises a pattern of distortion product otoacoustic emission growth with increasing stimulus level at a given frequency.

9. The method of claim 1, wherein the determining includes analyzing the recorded otoacoustic emissions using sliding time analysis windows with a weighted least-squares fitting (WLSF) procedure.

10. The method of claim 9, wherein the sliding time analysis windows are between 100-300 milliseconds in length, and wherein there is an overlap between windows.

11. A system for conducting an otoacoustic emission test, the system comprising:
a signal generator that emits a stimulus tone that is to be received by a user, wherein the stimulus tone comprises a swept level stimuli; and
a computing device operatively coupled to the signal generator, wherein the computing device includes:
a memory that stores otoacoustic emissions generated by an inner ear of the user, wherein the otoacoustic emissions are in response to the stimulus tone; and
a processor operatively coupled to the memory, wherein the processor determines one or more health characteristics of the inner ear of the user based at least in part on the otoacoustic emissions.

12. The system of claim 11, wherein the swept level stimuli simultaneously includes a plurality of distinct frequencies.

13. The system of claim 11, wherein the stimulus tone comprises a pair of stimulus tones that have a fixed frequency ratio.

14. The system of claim 11, wherein the fixed frequency ratio is in a range between 1.1 and 1.3.

15. The system of claim 11, wherein the processor is further configured to calibrate the stimulus tone using a forward pressure level (FPL) technique.

16. The system of claim 11, further comprising a measurement probe configured to record the otoacoustic emissions generated by the inner ear of the user.

17. The system of claim 11, wherein the one or more health characteristics include an amount of cochlear aging in the user.

18. The system of claim 11, wherein the one or more health characteristics include a growth function for the inner ear of the user.

19. The system of claim 18, wherein the growth function comprises a pattern of distortion product otoacoustic emission growth with increasing stimulus level at a given frequency.

20. The system of claim 11, wherein the processor analyzes the otoacoustic emissions using sliding time analysis windows with a weighted least-squares fitting (WLSF) procedure to determine the one or more health characteristics.
